(19) United States
(12) Patent Application Publication
(10) Pub. No.: US 2025/0342960 A1
(43) Pub. Date: Nov. 6, 2025

(54) INFORMATION PROCESSING SYSTEM AND INFORMATION PROCESSING METHOD

(71) Applicants: SHIONOGI & CO., LTD., Osaka-shi, Osaka (JP); KYOTO UNIVERSITY, Kyoto-shi, Kyoto (JP); NATIONAL UNIVERSITY CORPORATION NARA INSTITUTE OF SCIENCE AND TECHNOLOGY, Ikoma-shi, Nara (JP)

(72) Inventors: Yuki YOSHIDA, Osaka-shi, Osaka (JP); Ryo KIGUCHI, Osaka-shi, Osaka (JP); Satoki FUJITA, Osaka-shi, Osaka (JP); Ayano HATA, Osaka-shi, Osaka (JP); Toshiaki FURUKAWA, Kyoto-shi, Kyoto (JP); Aran TAJIKA, Kyoto-shi, Kyoto (JP); Junichiro YOSHIMOTO, Ikoma-shi, Nara (JP)

(21) Appl. No.: 18/866,885

(22) PCT Filed: May 18, 2023
(86) PCT No.: PCT/JP2023/018543

§ 371 Date: Nov. 18, 2024

(30) Foreign Application Priority Data
May 19, 2022 (JP) 2022-082495

(51) Int. Cl. G16H 50/20

(52) U.S. Cl. CPC G16H 50/20

(57) ABSTRACT

An object is to accurately predict recurrence and worsening of depression symptoms in advance. An information processing system (100) includes: a clustering unit (52) that inputs behavior record information of a user (U) to a clustering model that classifies behavior patterns into a plurality of clusters, and classifies behavior patterns of the user (U) into any of the plurality of clusters; a first feature amount generation unit (53) that generates a first feature amount indicating an activity state of the user U; and an estimation unit (55) that estimates a magnitude of psychological stress of the user (U) on the basis of object person information including attribute information of an object person, a cluster into which the behavior patterns of the object person are classified, and the first feature amount.

### TECHNICAL FIELD

The present invention relates to an information processing system, an information processing method, and the like.

### BACKGROUND ART

Non-patent Document 1 discloses a technique of creating a feature amount from biometric data obtained from a wearable device and predicting the presence or absence of depression symptom and a HAM-D score that is one of depression symptom evaluation indexes.

Non-patent Document 2 discloses a panel VAR model considering a relationship between a risk factor of depression recurrence and deterioration of a mental health condition and an estimation result thereof.

### PRIOR ART REFERENCES

### Non-Patent Document

[Non-patent Document 1] Tazawa et al. “Evaluating depression with multimodal wristband-type wearable device: screening and assessing patient severity utilizing machine-learning”, Heliyon 6, e03274, 2020
[Non-patent Document 2] Kumagai et al. “Predicting recurrence of depression using lifelog data: an explanatory feasibility study with a panel VAR approach”, BMC Psychiatry 19:391, 2019

### SUMMARY OF THE INVENTION

### Problems to be Solved by the Invention

Even if depression is once ameliorated by treatment, the probability of recurrence is high, and a period of sick/injured tends to be prolonged, and this causes a decrease in labor productivity as a social problem. If the treatment can be started before the depression symptoms recur or worsen, a high therapeutic effect can be expected. There is a great interest in techniques for accurately predicting recurrence and worsening of depression symptoms.

An object of one aspect of the present invention is to realize an information processing system and an information processing method capable of accurately predicting recurrence and worsening of depression symptoms in advance.

### Means for Solving the Problem

In order to solve the above problem, an information processing system according to one aspect of the present invention comprises: a clustering unit that inputs, to a clustering model that classifies behavior patterns of a plurality of depression patients into a plurality of clusters, behavior record information in which time of behavior performed by an object person during a first period is recorded for each behavior type, and classifies behavior patterns of the object person into any of the plurality of clusters; a first feature amount generation unit that, based on measurement information including an activity amount and a sleep time of the object person measured during a second period, generates a first feature amount indicating an activity state of the object person for each partial period included in the second period; and an estimation unit that estimates, for each of the plurality of clusters, a magnitude of psychological stress of the object person after the second period based on object person information including attribute information of the object person, a cluster into which the object person is classified, and the first feature amount.

In order to solve the above problem, an information processing method according to one aspect of the present invention is an information processing method by a computer, the information processing method comprising: a clustering step of inputting, to a clustering model that classifies behavior patterns of a plurality of depression patients into a plurality of clusters, behavior record information in which time of behavior performed by an object person during a first period is recorded for each behavior type, and classifies behavior patterns of the object person into any of the plurality of clusters; a first feature amount generation step of, based on measurement information including an activity amount and a sleep time of the object person measured during a second period, generating a first feature amount indicating an activity state of the object person for each partial period included in the second period; and an estimation step of estimating, for each of the plurality of clusters, a magnitude of psychological stress of the object person after the second period based on object person information including attribute information of the object person, a cluster into which the object person is classified, and the first feature amount.

The information processing apparatus according to each aspect of the present invention may be implemented by a computer. In this case, a control program for an information processing apparatus that causes a computer to implement the information processing apparatus by operating the computer as each unit (software element) included in the information processing apparatus, and a computer-readable recording medium recording the control program are also included in the scope of the present invention.

### Effect of the Invention

According to one aspect of the present invention, it is possible to accurately predict recurrence and worsening of depression symptoms in advance.

### BRIEF DESCRIPTION OF THE DRAWINGS

FIG. 1 is a block diagram illustrating an example of a configuration of an information processing system according to a first embodiment of the present invention.

FIG. 2 is a flowchart illustrating an example of a flow of processing by an information processing apparatus in the information processing system.

FIG. 3 is a diagram illustrating an example of processing of generating a first feature amount.

FIG. 4 is a diagram illustrating an example of processing of generating a second feature amount.

FIG. 5 is a block diagram illustrating an example of a configuration of an information processing system according to a second embodiment of the present invention.

FIG. 6 is a block diagram illustrating an example of a configuration of an information processing system according to a third embodiment of the present invention.

FIG. 7 is a block diagram illustrating an example of a configuration of an information processing system according to a fourth embodiment of the present invention.

FIG. 8 is a table summarizing a correspondence relationship between a category predicted by a K6 score estimated using an estimation model as an example of the present invention and an actual category classified based on an actual K6 score of a depression patient.

FIG. 9 is a diagram illustrating an ROC curve created based on a category predicted by the K6 score estimated using the estimation model and an actual category classified based on the actual K6 score of a depression patient.

FIG. 10 is a diagram illustrating an ROC curve created based on a category predicted by the K6 score estimated using the estimation model and an actual category classified based on the actual K6 score of a depression patient.

FIG. 11 is a table showing values of AUC calculated using each graph illustrated in FIGS. 9 and 10.

### MODE FOR CARRYING OUT THE INVENTION

### First Embodiment

Hereinafter, an embodiment of the present invention will be described in detail.

### (Configuration of Information Processing System 100)

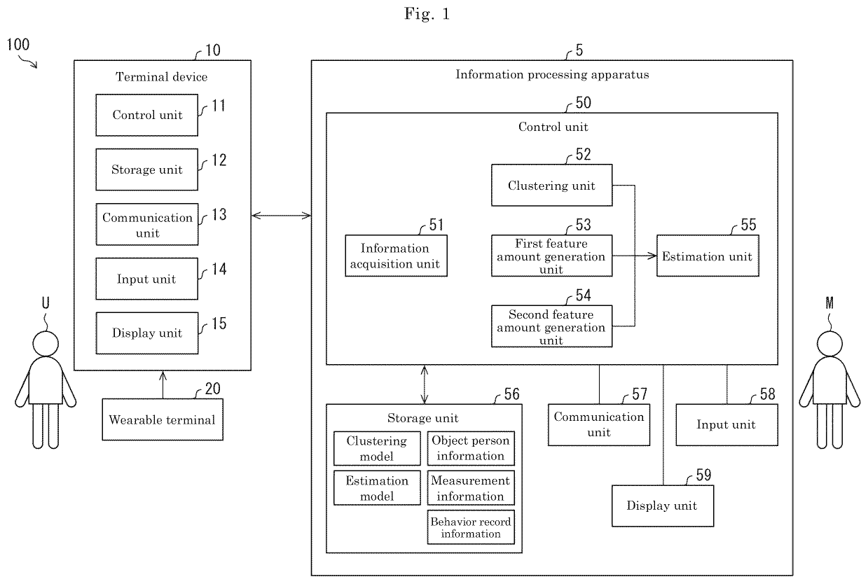

FIG. 1 is a block diagram illustrating an example of a configuration of an information processing system 100 according to the present embodiment. The information processing system 100 estimates a magnitude of the psychological stress of a user U on the basis of information (hereinafter, it is referred to as user information) such as an activity state of the user U, attribute information of the user U, and a behavior pattern of the user U. More specifically, the information processing system 100 estimates the magnitude of the psychological stress of the user U in order to detect in advance that there is a risk of recurrence or relapse of depression for the user U as an object person who has previously developed depression.

As illustrated in FIG. 1, the information processing system 100 may include a terminal device 10 and a wearable terminal 20 used by the user U, and an information processing apparatus 5. In the information processing system 100, the user information is transmitted from the terminal device 10 to the information processing apparatus 5 used by a medical practitioner M such as an attending physician of the user U, for example, and the information processing apparatus 5 estimates the magnitude of the psychological stress of the user U. Note that, in the present embodiment, the information processing apparatus 5 will be described as being used by the medical practitioner M, but the present invention is not limited thereto. The information processing apparatus 5 may be used by the user U or may be used by a family member of the user U.

First, a method of acquiring user information will be described. As illustrated in FIG. 1, the user information may be acquired by at least one of the terminal device 10 and the wearable terminal 20 carried by the user U.

The terminal device 10 may be a computer such as a smartphone or a tablet terminal. The terminal device 10 includes a control unit 11 that integrally controls each unit of the terminal device 10, a storage unit 12 that stores various data used by the terminal device 10, a communication unit 13 for the terminal device 10 to communicate with other devices, an input unit 14 that receives an input operation to the terminal device 10, and a display unit 15 that displays various types of information.

The terminal device 10 may have installed therein application software (hereinafter, referred to as an application) for receiving input of information indicating a behavior pattern of the user U and storing the information in the storage unit 12. The terminal device 10 may transmit information indicating the input behavior pattern to the information processing apparatus 5 or the like via the communication unit 13. The terminal device 10 may receive, via the input unit 14, the input of the user U regarding the behavior performed by the user U and the time when the user U performed the behavior via the application. The behavior of the user U may be classified into a plurality of items. For example, in a case where the behavior of the user U is classified into 16 items, the items may include “sleep”, “meal/snack”, “bath”, “work/study”, “average viewing time of media such as TV and DVD”, and the like. Note that, in one aspect of the present invention, at least part of the information indicating the behavior pattern of the user U may not be input by the user U. For example, it may be detected by a sensor that the user U is viewing a medium such as a TV or a DVD, and the sensor may calculate an “average viewing time of the medium such as a TV or a DVD” on the basis of a detection result and output the average viewing time to the information processing apparatus 5.

The wearable terminal 20 is a device worn on the body of the user U. The wearable terminal 20 has a function of measuring data related to an activity state of the user U. Here, the activity state may be the number of steps, calorie consumption, sleep time, conversation time, pulse rate, skin temperature, irradiated ultraviolet level, and the like. The wearable terminal 20 may be configured to output information such as measured data to the terminal device 10. The measurement data may include an activity amount and the sleep time of the user U. The wearable terminal 20 may be, for example, a wearable terminal worn on the head, neck, wrist, finger, chest, abdomen, ankle, or the like of the user U.

The terminal device 10 outputs, to the information processing apparatus 5 via the communication unit 13, information (hereinafter, also referred to as behavior record information) in which the time of the behavior performed by the user U is recorded for each behavior type, the information being input to the terminal device 10 by the user U via the application, and information (hereinafter, measurement information) regarding the activity state of the user U measured by the wearable terminal 20. In one aspect of the present invention, the wearable terminal 20 may directly output the measurement information to the information processing apparatus 5 without passing through the terminal device 10.

The information processing apparatus 5 may be a computer. The information processing apparatus 5 estimates the magnitude of the psychological stress of the user U. As illustrated in FIG. 1, the information processing apparatus 5 includes a control unit 50 that integrally controls each unit of the information processing apparatus 5, a storage unit 56 that stores various data used by the information processing apparatus 5, a communication unit 57 for the information processing apparatus 5 to communicate with other apparatuses, an input unit 58 that receives an input operation to the information processing apparatus 5, and a display unit 59 that displays various types of information. The control unit 50 includes an information acquisition unit 51, a clustering unit 52, a first feature amount generation unit 53, a second feature amount generation unit 54, and an estimation unit 55.

The information acquisition unit 51 acquires information regarding the user U from the terminal device 10 via the communication unit 57. The information acquisition unit 51 acquires attribute information of the user U as the information regarding the user U. Specifically, the attribute information of the user U is information including the gender, the educational background, the working style, the marital status, the age, the initial onset age of depression, the number of times of onset of depression, and the like. The information acquisition unit 51 stores object person information including the acquired attribute information in the storage unit 56.

Furthermore, the information acquisition unit 51 may acquire the behavior record information and the measurement information output from the terminal device 10. For example, the behavior performed by the user U may be recorded in the terminal device 10 by the user U himself/herself via the application. In this case, the information acquisition unit 51 can acquire the behavior record information from the terminal device 10 via the communication unit 57. However, the method by which the information acquisition unit 51 acquires the behavior record information is not limited thereto. For example, the information acquisition unit 51 may acquire the behavior record information from a paper medium in which the user U records his/her behavior record. In this case, the behavior record stored in the paper medium may be input to the information processing apparatus 5 by the input unit 58 by the medical practitioner M. Alternatively, the information acquisition unit 51 may have a known optical character recognition (OCR) function, and may be configured to directly read the behavior record stored in a paper medium. The information acquisition unit 51 stores the acquired behavior record information and measurement information in the storage unit 56.

The clustering unit 52 classifies the behavior patterns of the user U into any of a plurality of clusters created in advance by classifying the behavior patterns of a plurality of depression patients on the basis of the behavior record information stored in the storage unit 56. Details of a specific classification method by the clustering unit 52 will be described later.

The first feature amount generation unit 53 generates a feature amount (hereinafter, the feature amount is referred to as a first feature amount) of the measurement information stored in the storage unit 56. The first feature amount is a feature amount indicating an activity state of the user U. Details of the first feature amount and a method of generating the first feature amount by the first feature amount generation unit 53 will be described later.

The second feature amount generation unit 54 generates a feature amount (hereinafter, the feature amount is referred to as a second feature amount) of the behavior record information stored in the storage unit 56. The second feature amount is a feature amount indicating a behavior pattern of the user U. Details of the second feature amount and a method of generating the second feature amount by the second feature amount generation unit 54 will be described later.

The estimation unit 55 estimates the magnitude of the psychological stress of the user U. Specifically, the estimation unit 55 estimates the magnitude of the psychological stress of the user U using an estimation model prepared for the cluster in which the behavior pattern of the user U is classified by the clustering unit 52 among the estimation models prepared for each of the plurality of clusters. The estimation model may be stored in the storage unit 56 in advance. The estimation model is a model in which a plurality of variables such as background information (for example, an age, an age at onset, a working situation, and the like) of the object person, a weekly average and a standard deviation of the measurement information and the behavior record information, an absenteeism status, a correlation coefficient between a skin temperature and an irradiated ultraviolet ray level, the first feature amount generated by the first feature amount generation unit 53, and the second feature amount generated by the second feature amount generation unit 54 are used as explanatory variables, and the magnitude of the psychological stress is used as an objective variable. The index indicating the magnitude of the psychological stress is not particularly limited, and conventionally known K6 score, PHQ-9, HAM-D, and the like can be used. In a case where the K6 score is used as the objective variable, the estimation unit 55 may set the objective variable so as to classify the objective variable into a plurality of classes on the basis of the value of the K6 score. For example, the estimation unit 55 may set the objective variable to classify K6 scores into a plurality of classes, such as class 0 for K6 scores less than 5, class 1 for K6 scores 5 or more and less than 9, class 2 for K6 scores 9 or more and less than 13, and class 3 for K6 scores of 13 or more. Details of a method of estimating the magnitude of the psychological stress by the estimation unit 55 will be described later.

### (Example of Processing in Information Processing System 100)

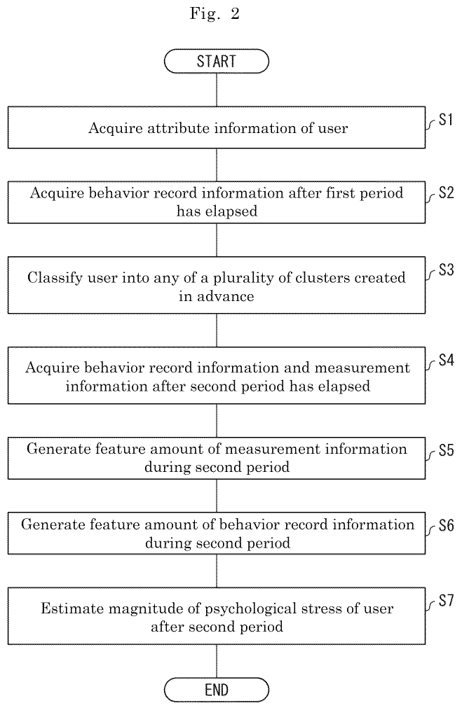

Next, an example of a flow of processing in the information processing system 100 according to the present embodiment will be described. FIG. 2 is a flowchart illustrating an example of a flow of processing by the information processing apparatus 5 in the information processing system 100. When the service using the information processing system 100 is started, as illustrated in FIG. 2, first, the information acquisition unit 51 of the information processing apparatus 5 acquires the attribute information of the user U (step S1: object person information acquisition step). The information acquisition unit 51 may acquire the attribute information input to the terminal device 10 by the user U via the communication unit 57. Alternatively, the attribute information may be input to the information processing apparatus 5 by the medical practitioner M. In this case, a predetermined questionnaire asking the attribute information of the user U may be performed in advance, and the medical practitioner M may input the attribute information to the information processing apparatus 5 on the basis of an answer to the questionnaire.

Next, the terminal device 10 starts acceptance of the behavior record performed by the user U via the application. In other words, the user U starts inputting the behavior performed by the user U using the application to the terminal device 10. Furthermore, the wearable terminal 20 starts measuring data regarding the activity state of the user U. The wearable terminal 20 outputs the measured measurement information to the terminal device 10.

When a predetermined period (hereinafter, the period is referred to as a first period) such as two weeks has elapsed since the input of the behavior pattern of the behavior by the user U to the terminal device 10 and the measurement of the data regarding the activity state of the user U by the wearable terminal 20 are started, the behavior record information recorded in the period is transmitted from the terminal device 10 to the information processing apparatus 5, and the information acquisition unit 51 of the information processing apparatus 5 acquires the behavior record information (step S2).

Next, the clustering unit 52 classifies the behavior patterns of the user U into any of a plurality of clusters created in advance by classifying the behavior patterns of a plurality of depression patients on the basis of the behavior record information during the first period acquired by the information acquisition unit 51 (step S3: clustering step). A clustering model for classification into the plurality of clusters is created in advance and stored in the storage unit 56.

Here, a method of creating the clustering model will be described. In the creation of the clustering model, first, a behavior pattern in a first period (for example, 14 days) is acquired for each of a plurality of depression patients. Note that the behavior pattern is a behavior pattern for a behavior performed in a period in which depression does not occur in the plurality of depression patients. Next, for each depression patient, an average value per day of each behavior pattern is calculated. Next, factor analysis is performed on the calculated average value per day of the behavior pattern of each depression patient, and a clustering model for classifying into a plurality of classification types is created based on a result of the factor analysis. The clustering model may be created using, for example, the k-means method.

The clustering unit 52 classifies the behavior pattern of the user U into any of the plurality of clusters by inputting the behavior record information in the first period into the clustering model created in advance by the above method and stored in the storage unit 56.

When a predetermined period (hereinafter, the period is referred to as a second period and a case where the second period is six weeks will be described) such as 6 to 8 weeks elapses from the start of the input of the behavior pattern of the behavior by the user U to the terminal device 10 and the measurement of the data regarding the activity state of the user U by the wearable terminal 20, the behavior record information in the second period and the measurement information in the second period are transmitted from the terminal device 10 to the information processing apparatus 5, and the information acquisition unit 51 of the information processing apparatus 5 acquires the information (step S4).

Next, the first feature amount generation unit 53 generates a feature amount (that is, the first feature amount) of the measurement information (that is, the measurement information measured by the wearable terminal 20 during the second period) for the measurement information during the second period (step S5, first feature amount generation step). Specifically, the first feature amount generation unit 53 first calculates the following numerical values (variables) on each day for each item included in the measurement information measured by the wearable terminal 20.
Calories consumed and the number of steps: Total amount, standard deviation per hour, maximum value per hour.
Pulse rate upon waking up and pulse rate during sleep: Median, standard deviation.
Conversation time: Total conversation time.
Skin temperature and UV level: Total per day, average value per hour, standard deviation per hour, maximum value, 75% tile value, 90% tile value, 95% tile value, 99% tile value.

The first feature amount generation unit 53 calculates an average value of the calculated numerical values for each week included in the second period. For each calculated average value, the first feature amount generation unit 53 generates, as the first feature amount, lugs of one week, two weeks, three weeks, and four weeks before each week (each partial period).

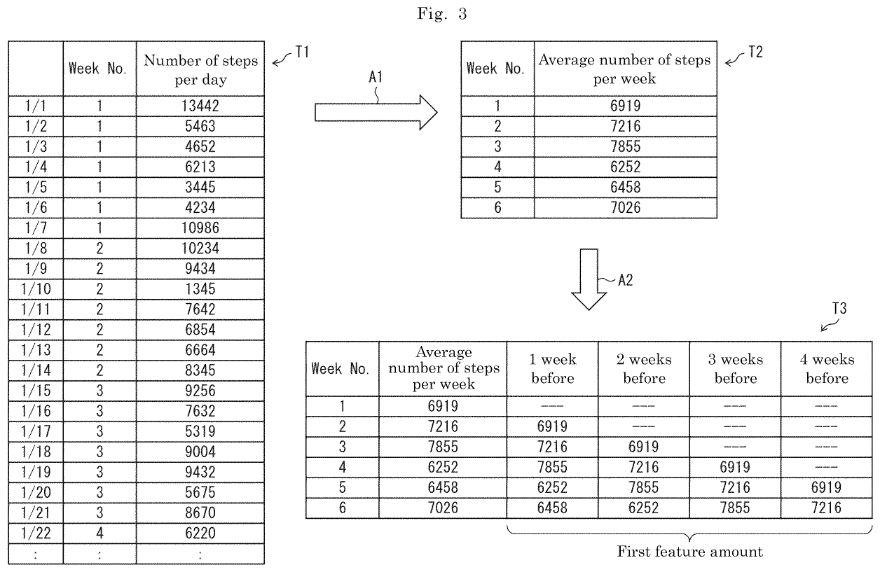

Here, an example of processing in which the first feature amount generation unit 53 generates the first feature amount will be described with reference to FIG. 3. FIG. 3 is a diagram illustrating an example of processing of generating the first feature amount. In particular, FIG. 3 illustrates an example of processing in a case where the first feature amount regarding the total of the number of steps per day is generated. As shown in a table denoted by reference signs T1 in FIG. 3, here, January 1 will be described as start dates of the first period and the second period. First, the first feature amount generation unit 53 calculates the total number of steps in each day from the measurement information (see a table denoted by reference sign T1 in FIG. 3). Next, the first feature amount generation unit 53 calculates the average number of steps in the first week (January 1 to January 7), the second week (January 8 to January 14), the third week (January 15 to January 21), the fourth week (January 22 to January 28), the fifth week (January 29 to February 4), and the sixth week (February 5 to February 11) (see the table indicated by reference sign T2 in FIG. 3) from the calculated data of the number of steps per day (processing indicated by arrow A1 in FIG. 3). Then, as illustrated in a table indicated by reference sign T3 in FIG. 3, the first feature amount generation unit 53 uses the calculated average number of steps in one week to generate, for each of the first to sixth weeks, lugs of one week before, two weeks before, three weeks before, and four weeks before as the first feature amount (processing indicated by arrow A2 in FIG. 3).

Note that the type of the variables described above is an example, and it is not always necessary to use the feature amounts for all the variables as the second feature amount, and feature amounts for other variables may be used.

Next, the second feature amount generation unit 54 generates a feature amount (that is, the second feature amount) of the behavior record information for the behavior record information during the second period (step S6, second feature amount generation step). Specifically, the second feature amount generation unit 54 first calculates the following numerical values (variables) for each item of the behavior performed by the user U during the second period for each week included in the second period.
Mean time.
Standard deviation.
The number of days falling outside an upper limit of the 99% confidence interval calculated based on the data during a period from the collection start date of the behavior record information to 14 days after the start (that is, the first period).
The number of days falling outside the lower limit of the 99% confidence interval calculated based on the data for the first period.
The number of days falling outside the 99% confidence interval calculated based on the data for the first period.
The number of days falling outside the upper limit of the 95% confidence interval calculated based on the data for the first period.
The number of days falling outside the lower limit of the 95% confidence interval calculated based on the data for the first period.
The number of days falling outside the 95% confidence interval calculated based on the data for the first period.

For each calculated variable, the second feature amount generation unit 54 generates, as the second feature amount, lugs one week before, two weeks before, three weeks before, and four weeks before each week.

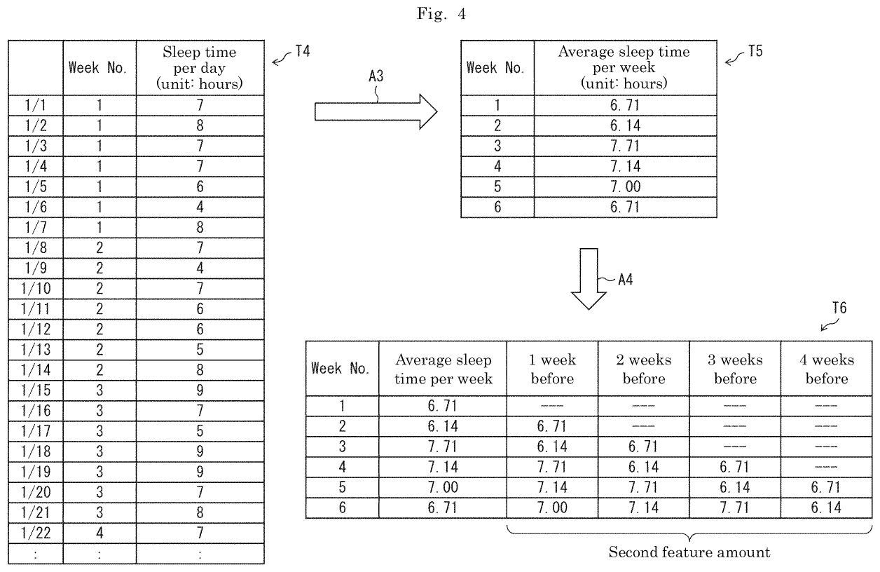

Here, an example of processing in which the second feature amount generation unit 54 generates the second feature amount will be described with reference to FIG. 4. FIG. 4 is a diagram illustrating an example of processing of generating the second feature amount. In particular, FIG. 4 illustrates an example of processing of generating the second feature amount regarding the average time for the sleep time as an example of the behavior type. First, the second feature amount generation unit 54 calculates an average sleep time in each week of the first to sixth weeks from the data of the sleep time per day shown in a table denoted by reference sign T4 in FIG. 4 as shown in a table denoted by reference sign T5 in FIG. 4 (processing indicated by arrow A3 in FIG. 3). Then, the second feature amount generation unit 54 generates, as a second feature amount, lugs of one week before, two weeks before, three weeks before, and four weeks before for the first to sixth weeks of the calculated average sleep as illustrated in a table denoted by reference sign T6 in FIG. 4 (processing indicated by an arrow A4 in FIG. 3).

Note that the type of the variables described above is an example, and it is not always necessary to use the feature amounts for all the variables as the second feature amount, and feature amounts for other variables may be used. For example, the second feature amount generation unit 54 may obtain an absenteeism rate, the number of meals, and the time of having dinner as variables from the behavior record information in the second period, and generate, as the second feature amount, a lag of the variables one week before, two weeks before, three weeks before, and four weeks before each week for these variables.

Next, the estimation unit 55 estimates the magnitude of the psychological stress of the user U after the second period (step S7, estimation step). A specific estimation method will be described below. First, the storage unit 56 stores an estimation model prepared for each of a plurality of clusters classified by the clustering model described above.

The estimation model is created as follows. That is, it is confirmed which cluster among the plurality of clusters corresponds to each of the plurality of depression patients targeted at the time of creating the clustering model. Here, for simplification, an example of classification into two clusters of a first cluster and a second cluster by the clustering model will be described. Next, using the same method as the method performed by the first feature amount generation unit 53 and the second feature amount generation unit 54, the first feature amount and the second feature amount are generated for a plurality of depression patients whose behavior patterns are classified into the first cluster. Then, machine learning is performed using teacher data in which attribute information of each depression patient whose behavior pattern is classified into the first cluster, and a plurality of variables including the calculated first feature amount and second feature amount are used as explanatory variables, and the magnitude of psychological stress is used as an objective function. As a result, an estimation model for the first cluster is created. Furthermore, a first feature amount and a second feature amount are generated for a plurality of depression patients whose behavior patterns are classified into the second cluster, and machine learning is performed using teacher data in which attribute information of each depression patient whose behavior patterns are classified into the second cluster, and a plurality of variables including the calculated first feature amount and second feature amount are used as explanatory variables, and the magnitude of psychological stress is used as an objective function, whereby an estimation model for the second cluster is created. As described above, the estimation model of each cluster is created by performing machine learning using the teacher data in which the plurality of variables including the attribute information, the first feature amount, and the second feature amount of the depression patient belonging to each cluster are used as the explanatory variables and the magnitude of the psychological stress is used as the objective function.

A model of machine learning used to create the estimation model is not particularly limited, but for example, Xgboost, lightGBM, or the like can be used. Xgboost is an abbreviation of eXtreme Gradient Boosting, and is a method in which ensemble learning called gradient boosting and a decision tree are combined. lightGBM is a machine learning framework for gradient boosting based on a decision tree algorithm.

Furthermore, the number of types of explanatory variables used as teacher data may be reduced by performing feature amount engineering using BORUTA or the like. BORUTA is a method of selecting an explanatory variable by comparing whether the importance is significantly higher than the noise based on the feature amount importance.

The estimation unit 55 estimates the magnitude of the psychological stress of the user U after the second period by inputting the object person information including the attribute information of the user U, the first feature amount generated by the first feature amount generation unit 53, and the second feature amount generated by the second feature amount generation unit 54 to the estimation model prepared for the cluster in which the behavior pattern of the user U is classified by the clustering unit 52. In other words, the estimation unit 55 estimates the magnitude of the psychological stress of the user U after the second period by inputting the first feature amount generated by the first feature amount generation unit 53 and the second feature amount generated by the second feature amount generation unit 54 to the estimation model prepared for the cluster in which the behavior pattern of the user U is classified by the machine learning using the teacher data in which the object person information, the first feature amount, and the second feature amount are set as the explanatory variables and the magnitude of the psychological stress is set as the objective function.

The information processing apparatus 5 may cause the display unit 59 to display the magnitude of the psychological stress estimated by the estimation unit 55.

Although FIG. 2 illustrates the processing of acquiring the object person information, the measurement information, and the behavior record information, the processing is not limited thereto. For example, in a case where the object person information, the measurement information, and the behavior record information acquired in advance are stored in the storage unit 56, the information processing apparatus 5 may perform the steps after step S3.

Note that, since the clustering model is a model including the behavior record information of the user U as an explanatory variable, the clustering model can be classified into clusters reflecting the behavior pattern of the user U. Therefore, it is not essential that the estimation model includes the second feature amount as the explanatory variable.

That is, the estimation model of one aspect of the present invention may be an estimation model machine-learned using teacher data in which a plurality of variables including the object person information and the first feature amount but not including the second feature amount are used as explanatory variables and the magnitude of the psychological stress is used as an objective function. In this case, the estimation unit 55 estimates the magnitude of the psychological stress of the user U after the second period by inputting the attribute information of the user U acquired by the information acquisition unit 51 and the first feature amount generated by the first feature amount generation unit 53. That is, the estimation unit 55 may estimate the magnitude of the psychological stress of the user U after the second period without inputting the second feature amount. In this case, since the user U does not need to record his/her own behavior after the first period, the burden on the user U who uses the information processing system 100 can be reduced.

Furthermore, the estimation model of one aspect of the present invention may further include other variables as explanatory variables. For example, the estimation model of one aspect of the present invention may include, as an explanatory variable, an index such as PHQ-9 or BDI-II obtained by conducting a hearing survey for the user U by telephone or the like. In this case, the estimation unit 55 also inputs, to the estimation model, indices such as the PHQ-9 and the BDI-II obtained by the hearing survey performed on the user U during the second period.

### (Effects of Information Processing System 100)

In a case where depression symptoms recur or worsen, changes in behavior patterns and activity state are often seen as a sign of the recurrence or worsening. Furthermore, the changes in the behavior pattern and the activity state vary depending on the normal behavior pattern and activity state of the object person. Therefore, in order to accurately estimate the psychological situation of the object person, it is desirable to perform classification based on the tendency of the normal behavior pattern and activity state of the object person and then detect a change in the behavior pattern and the activity state.

As described above, in the information processing system 100, the clustering unit 52 inputs the behavior record information in which the time of the behavior performed by the object person during the first period is recorded for each behavior type to the clustering model that classifies the behavior patterns of the plurality of depression patients into the plurality of clusters. As a result, the information processing system 100 classifies the behavior pattern of the user U into any of a plurality of clusters. Furthermore, in the information processing system 100, the first feature amount generation unit 53 generates a first feature amount that is a feature amount indicating the weekly activity state of the user U on the basis of the measurement information during the second period. Furthermore, in the information processing system 100, the second feature amount generation unit 54 generates a second feature amount that is a feature amount indicating the weekly activity pattern of the user U on the basis of the behavior record information in the second period. Then, in the information processing system 100, the estimation unit 55 inputs the object person information including the attribute information of the user U, the first feature amount, and the second feature amount to the estimation model prepared for the cluster in which the behavior pattern of the user U is classified. As a result, the information processing system 100 estimates the magnitude of the psychological stress of the user U after the second period. As a result, the information processing system 100 can accurately estimate the magnitude of the psychological stress of the user U after the second period on the basis of the change caused in the behavior pattern and the average activity state of the user U in the second period.

By using the information processing system 100, for example, in a case where the magnitude of the estimated psychological stress is large, the medical practitioner M can urge the user U to visit the hospital. As a result, the user U can receive medical care at an early stage, and can receive treatment before the disease state deteriorates.

Furthermore, the information processing apparatus 5 may notify the user U of the estimated magnitude of the psychological stress via the communication unit 57. The method by which the information processing apparatus 5 notifies the user of the magnitude of the psychological stress may be as follows.
A web page for notifying each user of the magnitude of psychological stress is created, and access information for accessing the web page is notified to each user.
A display screen for notifying the user of the magnitude of the psychological stress is displayed on the display unit 15.

As a result, the user U can recognize the degree of deterioration of his/her own disease state after the second period.

The information processing system 100 may include a distribution server that distributes the clustering model and the estimation model used by the information processing apparatus 5. When the clustering model and the estimation model are distributed from the distribution server, the information processing apparatus 5 may update the clustering model and the estimation model stored in the storage unit 56 to the distributed clustering model and estimation model.

### (Modification)

In the information processing system 100 according to the first embodiment, the information processing apparatus 5 includes the clustering unit 52, the first feature amount generation unit 53, the second feature amount generation unit 54, and the estimation unit 55, but the information processing system 100 according to the present invention is not limited thereto. In the information processing system 100 according to one aspect of the present invention, the terminal device 10 may have a configuration including a part of the functions of the control unit 50 of the information processing apparatus 5. For example, the terminal device 10 may generate the first feature amount using the measurement information measured by the wearable terminal and output the generated first feature amount to the information processing apparatus 5. In this case, the estimation unit 55 may estimate the magnitude of the psychological stress of the user U after the second period by inputting the attribute information of the user U acquired by the information acquisition unit 51, the first feature amount generated by the terminal device 10, and the second feature amount generated by the second feature amount generation unit 54.

### Second Embodiment

Another embodiment of the present invention will be described below. Note that, for convenience of description, members having the same functions as the members described in the above embodiment are denoted by the same reference signs, and the description thereof will not be repeated.

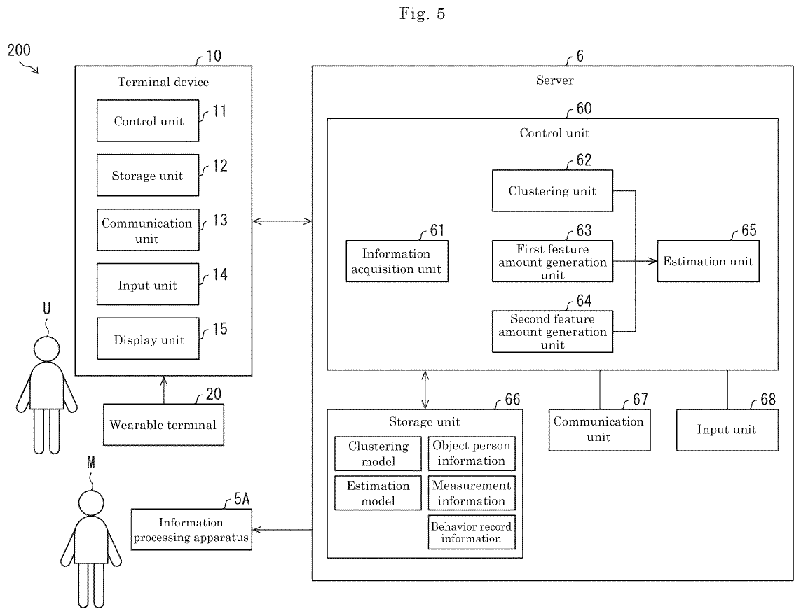

FIG. 5 is a block diagram illustrating an example of a configuration of an information processing system 200 according to the present embodiment. As illustrated in FIG. 5, the information processing system 200 may include a terminal device 10 and a wearable terminal 20 used by a user U, and a server 6. In the information processing system 200, the user information is transmitted from the terminal device 10 to the server 6, and the magnitude of the psychological stress of the user U is estimated by the server 6.

The server 6 estimates the magnitude of the psychological stress of the user U. The server 6 may be a computer. As illustrated in FIG. 5, the server 6 includes a control unit 60 that integrally controls each unit of the server 6, a storage unit 66 that stores various data used by the server 6, a communication unit 67 for the server 6 to communicate with other devices, and an input unit 68 that receives an input operation to the server 6. The control unit 60 includes an information acquisition unit 61 (object person information acquisition unit), a clustering unit 62, a first feature amount generation unit 63, a second feature amount generation unit 64, and an estimation unit 65.

The information acquisition unit 61 acquires information regarding the user U from the terminal device 10 via the communication unit 67. The information acquisition unit 61 acquires attribute information of the user U as the information regarding the user U. Furthermore, the information acquisition unit 61 may acquire the behavior record information and the measurement information output from the terminal device 10. The information acquisition unit 61 stores each acquired information in the storage unit 66.

The clustering unit 62 classifies the behavior patterns of the user U into any of a plurality of clusters created in advance by classifying the behavior patterns of a plurality of depression patients on the basis of the behavior record information acquired by the information acquisition unit 61. The method of classification by the clustering unit 62 is the same as the method by the clustering unit 52 in the first embodiment.

For the measurement information acquired by the information acquisition unit 61, the first feature amount generation unit 63 generates a feature amount (that is, the first feature amount) of the measurement information. A method of generating the first feature amount by the first feature amount generation unit 63 is similar to the method by the first feature amount generation unit 53 in the first embodiment.

The second feature amount generation unit 64 generates a feature amount (that is, the second feature amount) of the behavior record information for the behavior record information acquired by the information acquisition unit 61. A method of generating the second feature amount by the second feature amount generation unit 64 is similar to the method by the second feature amount generation unit 54 in the first embodiment.

The estimation unit 65 estimates the magnitude of the psychological stress of the user U. The estimation unit 65 inputs the attribute information of the user U, the first feature amount generated by the first feature amount generation unit 63, and the second feature amount generated by the second feature amount generation unit 64 to the estimation model prepared for the cluster in which the behavior pattern of the user U is classified by the clustering unit 62. As a result, the estimation unit 65 estimates the magnitude of the psychological stress of the user U after the second period. An estimation method by the estimation unit 65 is the same as the method by the estimation unit 55 in the first embodiment.

As described above, in the information processing system 200 according to the present embodiment, the server 6 estimates the magnitude of the psychological stress of the user U performed by the information processing apparatus 5 in the first embodiment. That is, in the information processing system 200, the estimation unit 65 estimates the magnitude of the psychological stress of the user U after the second period by inputting the object person information, the first feature amount, and the second feature amount including the attribute information of the user U to the estimation model prepared for the cluster in which the behavior pattern of the user U is classified by the clustering unit 62. As a result, the information processing system 200 can accurately estimate the magnitude of the psychological stress of the user U after the second period on the basis of the change caused in the behavior pattern and the average activity state of the user U in the second period.

In the information processing system 200, in a case where the magnitude of the psychological stress estimated by the server 6 is large, the information may be output to an information processing apparatus 5A possessed by a medical practitioner M via the communication unit 67. As a result, the medical practitioner M can urge the user U to visit the hospital. As a result, the user U can receive medical care at an early stage, and can receive treatment before the disease state deteriorates.

Furthermore, the server 6 may notify the user U of the estimated magnitude of the psychological stress via the communication unit 67. As a result, the user U can recognize the degree of deterioration of his/her own disease state after the second period.

### Third Embodiment

Another embodiment of the present invention will be described below. Note that, for convenience of description, members having the same functions as the members described in the above embodiment are denoted by the same reference signs, and the description thereof will not be repeated.

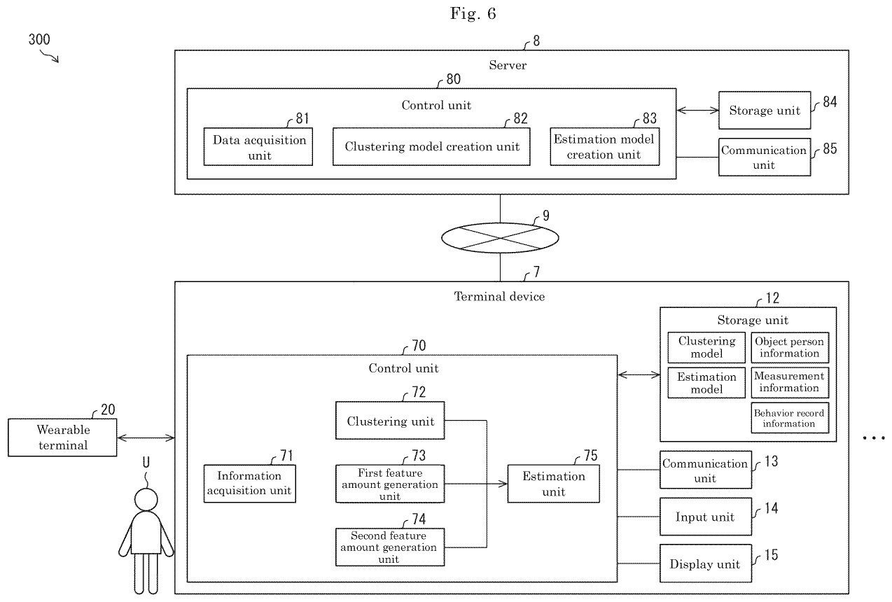

FIG. 6 is a block diagram illustrating an example of a configuration of an information processing system 300 according to the present embodiment. As illustrated in FIG. 6, the information processing system 300 may include a terminal device 7 and a wearable terminal 20 used by a user U, and a server 8. The terminal device 7 and the server 8 can communicate with each other via a network 9 such as the Internet. In the information processing system 300, the magnitude of the psychological stress of the user U is estimated by the terminal device 7.

The terminal device 7 may be a terminal device such as a smartphone or a tablet terminal. As illustrated in FIG. 6, the terminal device 7 includes a control unit 70 that integrally controls each unit of the terminal device 7, a storage unit 12, a communication unit 13, an input unit 14, and a display unit 15. The control unit 70 includes an information acquisition unit 71, a clustering unit 72, a first feature amount generation unit 73, a second feature amount generation unit 74, and an estimation unit 75.

The information acquisition unit 71 acquires measurement information regarding the activity state of the user U measured by the wearable terminal 20. Furthermore, the information acquisition unit 71 acquires behavior record information in which the time of the behavior performed by the user U is recorded for each behavior type, the behavior record information being recorded by the user U via the application.

The clustering unit 72 classifies the behavior patterns of the user U into any of a plurality of clusters by classifying the behavior patterns of a plurality of depression patients on the basis of the behavior record information acquired by the information acquisition unit 71. The clustering model for classification into a plurality of clusters is appropriately updated by the server 8 described later. Details of the update of the clustering model by the server 8 will be described later. Note that a classification method by the clustering unit 72 is the same as the method by the clustering unit 52 in the first embodiment except that the clustering model to be used is updated by the server 8.

For the measurement information acquired by the information acquisition unit 71, the first feature amount generation unit 73 generates a feature amount (that is, the first feature amount) of the measurement information. A method of generating the first feature amount by the first feature amount generation unit 73 is similar to the method by the first feature amount generation unit 53 in the first embodiment.

The second feature amount generation unit 74 generates a feature amount (that is, the second feature amount) of the behavior record information for the behavior record information acquired by the information acquisition unit 71. A method of generating the second feature amount by the second feature amount generation unit 74 is similar to the method by the second feature amount generation unit 54 in the first embodiment.

The estimation unit 75 estimates the magnitude of the psychological stress of the user U. The estimation unit 75 estimates the magnitude of the psychological stress of the user U after the second period by inputting the object person information including the attribute information of the user U, the first feature amount generated by the first feature amount generation unit 73, and the second feature amount generated by the second feature amount generation unit 74 to the estimation model prepared for the cluster in which the behavior pattern of the user U is classified by the clustering unit 72. The estimation model used for estimating the magnitude of the psychological stress is appropriately updated by the server 8 to be described later. Details of the update of the estimation model by the server 8 will be described later. Note that a method of estimating the magnitude of the psychological stress by the estimation unit 75 is the same as the method by the estimation unit 55 in the first embodiment except that the estimation model to be used is updated by the server 8.

The server 8 includes a control unit 80 that integrally controls each unit of the server 8, a storage unit 84 that stores various data used by the server 8, a communication unit 85 for the server 8 to communicate with other devices, and an input unit 86 that receives an input operation to the server 8. The control unit 80 includes a data acquisition unit 81, a clustering model creation unit 82, and an estimation model creation unit 83.

The data acquisition unit 81 acquires behavior record information and measurement information regarding a plurality of the users U who uses the information processing system 300. Specifically, the data acquisition unit 81 acquires the behavior record information and the measurement information acquired by the information acquisition unit 71 of the terminal device 7 possessed by the user U from each of the terminal devices 7 possessed by the plurality of users U. In this case, the data acquisition unit 81 may acquire identification information (for example, a user ID, a mail address, or the like) that can identify the user U together with various types of information. In this case, the server 8 may specify the user U and the terminal device 7 or the like possessed by the user U on the basis of the identification information, and provide information to the user U.

The clustering model creation unit 82 generates a clustering model based on the behavior record information (more specifically, the behavior pattern for 14 days for each of the plurality of users U) about the plurality of users U acquired by the data acquisition unit 81 and stored in the storage unit 84. The clustering model creation unit 82 may calculate an average value per day of each behavior pattern, perform factor analysis on the calculated average value per day of the behavior pattern of each depression patient, and create a clustering model for classifying into a plurality of classification types on the basis of a result of the factor analysis. The clustering model creation unit 82 may create a clustering model using, for example, the k-means method.

The clustering model creation unit 82 may create a clustering model every time a predetermined period elapses, or may create a clustering model every time behavior record information is additionally stored in the storage unit 84 for a predetermined number of people. The clustering model creation unit 82 may output the clustering model to the terminal device 7 every time the clustering model is created. When acquiring the clustering model from the clustering model creation unit 82, the terminal device 7 updates the clustering model stored in the storage unit 76 to the acquired clustering model.

The estimation model creation unit 83 creates an estimation model on the basis of the behavior record information and the measurement information for the plurality of users U acquired by the data acquisition unit 81 and stored in the storage unit 84. Specifically, the estimation model creation unit 83 confirms which cluster among the plurality of clusters corresponds to each of the plurality of users targeted when the clustering model creation unit 82 creates the clustering model. Here, an example of classification into two clusters of a first cluster and a second cluster by the clustering model will be described. Next, the estimation model creation unit 83 uses the same method as the method performed by the first feature amount generation unit 53 and the second feature amount generation unit 54 to generate the first feature amount and the second feature amount for a plurality of users whose behavior patterns are classified into the first cluster. Then, the estimation model creation unit 83 creates an estimation model for the first cluster by performing machine learning using teacher data in which attribute information of each user whose behavior pattern is classified into the first cluster and a plurality of variables including the calculated first feature amount and second feature amount are explanatory variables, and the magnitude of psychological stress is an objective function. Furthermore, similarly, the estimation model creation unit 83 creates an estimation model for the second cluster by generating a first feature amount and a second feature amount for a plurality of users whose behavior patterns are classified into the second cluster, and causing machine learning to be performed using teacher data in which attribute information of each user whose behavior patterns are classified into the second cluster and a plurality of variables including the calculated first feature amount and second feature amount are explanatory variables, and the magnitude of psychological stress is an objective function. As described above, the estimation model creation unit 83 creates the estimation model for each of the plurality of clusters by causing machine learning to be performed using the teacher data in which the plurality of variables including the attribute information, the first feature amount, and the second feature amount of the user belonging to each cluster are the explanatory variables and the magnitude of the psychological stress is the objective function.

A model of machine learning used to create the estimation model is not particularly limited, but for example, Xgboost, lightGBM, or the like can be used. Furthermore, in order to reduce the number of types of explanatory variables used as teacher data, feature amount engineering may be performed using BORUTA or the like.

Every time the clustering model creation unit 82 creates a clustering model, the estimation model creation unit 83 creates an estimation model for each of a plurality of clusters classified by the clustering model. When creating the estimation model, the estimation model creation unit 83 may create the estimation model using a variable including information regarding the user U who uses the created estimation model as a part of the explanatory variable and the objective variable. As a result, the magnitude of the psychological stress of the user U can be estimated more accurately.

The estimation model creation unit 83 outputs the created estimation model to the terminal device 7. The terminal device 7 updates the estimation model stored in the storage unit 76 to the estimation model output from the estimation model creation unit 83.

As described above, in the information processing system 300 according to the present embodiment, the terminal device 7 estimates the magnitude of the psychological stress of the user U performed by the information processing apparatus 5 in the first embodiment. That is, in the information processing system 200, the estimation unit 75 estimates the magnitude of the psychological stress of the user U after the second period by inputting the object person information including the attribute information of the user U, the first feature amount, and the second feature amount to the estimation model prepared for the cluster in which the behavior pattern of the user U is classified by the clustering unit 72. As a result, the information processing system 300 can accurately estimate the magnitude of the psychological stress of the user U after the second period on the basis of the change caused in the behavior pattern and the average activity state of the user U in the second period. As a result, the user U can recognize the degree of deterioration of his/her own disease state after the second period by confirming the magnitude of the psychological stress estimated by the terminal device 7.

Furthermore, in the information processing system 300, in a case where the magnitude of the psychological stress estimated by the terminal device 7 is large, the terminal device 7 may notify the medical practitioner M of the information via the communication unit 77. As a result, the medical practitioner M can urge the user U to visit the hospital. As a result, the user U can receive medical care at an early stage, and can receive treatment before the disease state deteriorates.

Moreover, in the information processing system 300, the clustering model used by the clustering unit 72 and the estimation model used by the estimation unit 75 can be updated as needed due to an increase in the number of users of the information processing system 300. As a result, it is possible to improve the accuracy of the plurality of clusters that classify the behavior patterns of the user U, and it is possible to improve the estimation accuracy of the estimation model for estimating the magnitude of the psychological stress of the user U. As a result, the magnitude of the psychological stress of the user U can be estimated more accurately.

In the information processing system 300 according to the third embodiment, the terminal device 7 includes the clustering unit 72, the first feature amount generation unit 73, the second feature amount generation unit 74, and the estimation unit 75, but the information processing system 300 of the present invention is not limited thereto. In the information processing system 300 according to one aspect of the present invention, the server 8 may be configured to include a part of the functions of the control unit 70 of the terminal device 7. For example, in the information processing system 300 according to one aspect of the present invention, the server 8 may have the function of the estimation unit 75. In this case, the estimation model created by the estimation model creation unit 83 may be stored in the storage unit 84. Then, the server 8 may estimate the magnitude of the psychological stress of the user U after the second period by inputting the attribute information (object person information) of the user U, the first feature amount, and the second feature amount output from the terminal device 7 to the estimation model stored in the storage unit 84. In this case, the server 8 may output the estimated magnitude of the psychological stress of the user U to the terminal device 7 via the network 9.

### Fourth Embodiment

Another embodiment of the present invention will be described below. Note that, for convenience of description, members having the same functions as the members described in the above embodiment are denoted by the same reference signs, and the description thereof will not be repeated.

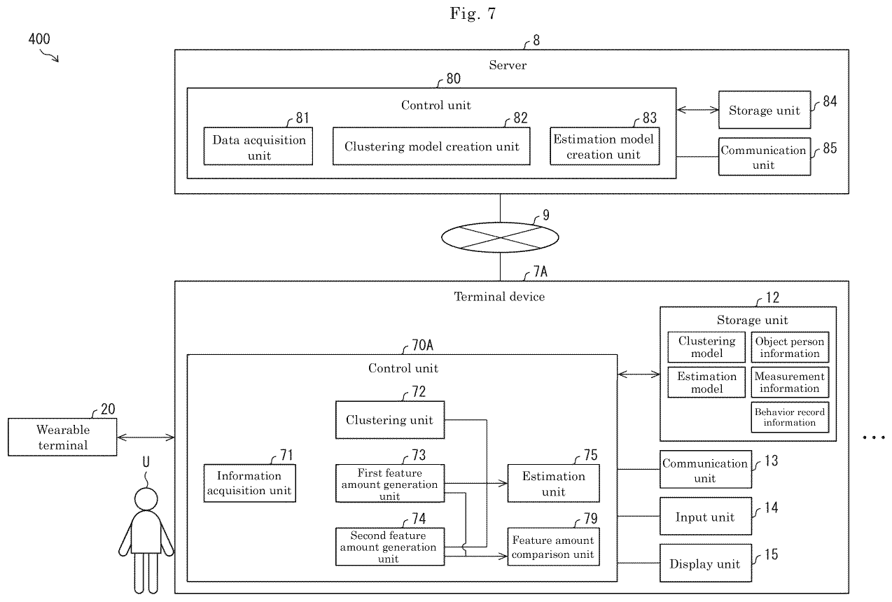

FIG. 7 is a conceptual diagram illustrating an example of a configuration of an information processing system 400 according to the present embodiment. As illustrated in FIG. 7, the information processing system 400 includes a terminal device 7A instead of the terminal device 7 in the third embodiment.

The terminal device 7A includes a control unit 70A instead of the control unit 70 in the seventh embodiment. The control unit 70A includes a feature amount comparison unit 79 in addition to the configuration of the control unit 70 in the third embodiment. With respect to the first feature amount and the second feature amount generated by a first feature amount generation unit 73 and a second feature amount generation unit 74, respectively, the feature amount comparison unit 79 compares the latest first feature amount and second feature amount with the first feature amount and the second feature amount in the period in which a clustering unit 72 acquires the information used when the behavior pattern of a user U is classified into any classification type. Specifically, the feature amount comparison unit 79 performs the above comparison by calculating the similarity (distance) between the first feature amount and the second feature amount using a method such as a root mean squared error (RMSE).

The terminal device 7A determines that there is a large change in the state of the user U in the latest period in a case where the latest first feature amount and second feature amount calculated by the feature amount comparison unit 79 are different from the first feature amount and the second feature amount in the period in which the clustering unit 72 has acquired the information used when the behavior pattern of the user U is classified into any classification type by exceeding a predetermined threshold value.

In a case where it is determined that there is a large change in the state of the user U in the latest period, the information processing system 400 performs a questionnaire for obtaining an index for achieving the magnitude of the psychological stress such as the PHQ-9 and the KG score for the user U, and determines whether or not the user U is in a healthy state. Then, in a case where the user U is in a healthy state, the clustering unit 72 classifies the behavior pattern of the user U into any of the plurality of clusters created in advance again on the basis of the behavior record information in which the time of the behavior performed by the user U in the latest two weeks is recorded for each type. On the other hand, in a case where the user U is in an abnormal state (for example, in a case where the user U re-develops depression, or the like), the questionnaire is conducted also after the next week. Then, at the time point when the user U is in a healthy state, the clustering unit 72 classifies the behavior pattern of the user U into one of the plurality of clusters created in advance again on the basis of the behavior record information in the period up to 2 weeks before the time point. Note that, in the method of estimating the magnitude of the psychological stress in the present invention, it is preferable to perform the estimation on the basis of information on the user in a state where the symptom is stable (in other words, a state that is not a depression state). Therefore, as described above, it is preferable to start using the information processing system 400 in the present embodiment again from the time point when it is determined from the questionnaire result that the user U is in a healthy state.

According to the above configuration, in a case where the behavior pattern of the user U greatly changes due to, for example, a change in lifestyle due to a job loss or a change from day duty to night duty due to a job change, the feature amount comparison unit 79 can detect that the change has occurred. As a result, the behavior pattern of the user U can be reclassified into an appropriate cluster by the clustering unit 72, and an estimation unit 75 can estimate the magnitude of the subsequent psychological stress of the user U using the estimation model prepared for the cluster into which the behavior pattern of the user U has been reclassified. As a result, even in a case where the behavior pattern of the user U greatly changes, the magnitude of the psychological stress of the user U can be estimated more accurately.

### EXAMPLES

Next, an example in which the estimation accuracy of the estimation method of one aspect of the present invention is verified will be described.

In this example, 89 depression patients were analyzed. First, for each depression patient over a year, behavior record information, measurement information measured by a wearable terminal worn on a wrist, and information of K6 score, PHQ-9, and BDI-II obtained by a telephone hearing survey were acquired. The behavior record information is information recorded by each depression patient via an application installed on a terminal device. The behavior record information is information recorded as to which of the items classified into 16 items (specifically, “sleep”, “rolling around, dazed”, “meal/snack”, “bath”, “work/study”, “movement/commuting/school”, “housework”, “child care/nursing care”, “shopping”, “hospital”, “interaction/association”, “sports/exercise”, “hobby/amusement/lesson”, “reading/newspaper/magazine”, “TV/DVD/music”, “others”) has been performed. The measurement information measured by the wearable terminal includes information such as the number of steps, calorie consumption, sleep time, conversation time, a pulse rate, a skin temperature, and an ultraviolet ray level to be irradiated.

In this example, for the behavior record information on 89 depression patients, 89 depression patients were clustered into two clusters of the first cluster and the second cluster using the k-means method. As a result, the number of depression patients whose behavior patterns were classified into the first cluster was 30, and the number of depression patients whose behavior patterns were classified into the second cluster was 59.

Next, for each item of the measurement information measured by the wearable terminal, variables such as a total amount, a standard sensor per hour, a median value, and a maximum value were calculated for each depression patient. Next, the average value for each week was calculated for the calculated variables, and the lugs one week before, two weeks before, three weeks before, and four weeks before each week were generated as first feature amounts.

Furthermore, for each item of the behavior record information, variables such as the average time, the standard deviation, and the number of days outside an upper limit of the 99% confidence interval calculated on the basis of the data from the collection start date of the behavior record information to 14 days after the start were calculated every week. Next, for each calculated variable, lugs one week before, two weeks before, three weeks before, and four weeks before each week were generated as second feature amounts.

Next, an estimation model for estimating the KG score as the magnitude of the psychological stress was created for each of the first cluster and the second cluster, and the estimation accuracy of the created estimation model was verified using the k-fold method. The estimation model was created by machine learning using, as explanatory variables, about 1,300 variables such as background information (for example, an age, an age at onset, a working situation, and the like) of the object person, a weekly average and standard deviation of measurement information and behavior record information, an absenteeism status, a correlation coefficient between a skin temperature and an irradiated ultraviolet level, the first feature amount, and the second feature amount, as explanatory variables, and an objective variable as a KG score.

Specifically, depression patient data was divided into four for each cluster, and an estimation model was created using ¾ of the data as teacher data. The estimation model was created as follows. First, for all data of depression patients of each cluster, variables used for creation of an estimation model were selected using BORUTA. Next, an estimation model having the selected variable as an explanatory variable and the KG score as an objective variable was created using Xgboost.

Next, the remaining ¼ data not used as teacher data was applied as test data to the estimation model created for each of the first cluster and the second cluster, the KG score of each depression patient was estimated, and each depression patient was classified into any of the following four categories.
Class 0: The KG score is less than 5
Class 1: The KG score is 5 or more and less than 9
Class 2: The KG score is 9 or more and less than 13
Class 3: The KG score is 13 or more.

Moreover, the above processing was further performed three times while replacing the teacher data and the data used as the test data.

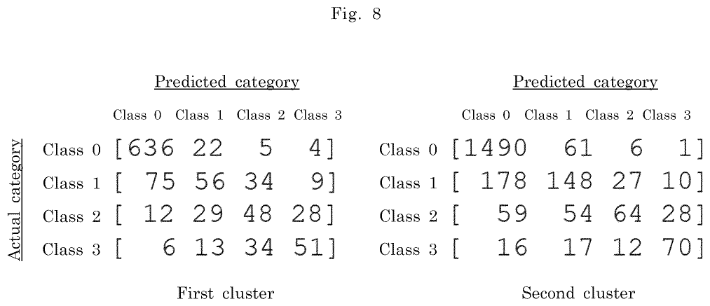

FIG. 8 is a table summarizing a correspondence relationship between a category predicted by the KG score estimated using the estimation model created and an actual category classified based on the actual KG score of the depression patient. Note that the numerical value illustrated in FIG. 8 is a numerical value obtained by adding the results of the above-described four times of processing.

Next, a weighted kappa coefficient was calculated with respect to the numerical values in the table illustrated in FIG. 8. As a result, the weighted kappa coefficient of the first cluster was 0.7818, and the weighted kappa coefficient of the second cluster was 0.7151. Note that, in a case where the first cluster and the second cluster were combined, the weighted kappa coefficient of the second cluster was 0.7147. As a criterion for evaluating the weighted kappa coefficient, Landis and Koch's criteria are known. According to the criteria of Landis and Koch, when the weighted kappa coefficient is less than 0, it is considered as “No agreement”, when 0.00 to 0.20, it is considered as “Slight”, when 0.21 to 0.40, it is considered as “Fair”, when 0.41 to 0.60, it is considered as “Moderate”, when 0.61 to 0.80, it is considered as “Substantial”, and when 0.81 to 1.00, it is considered as “Almost perfect”. As described above, in a case where the estimation model created in the present modification is used, the weighted kappa coefficient becomes “almost perfect”, and it is proved that the estimation accuracy of the estimation model created in the present modification is high.

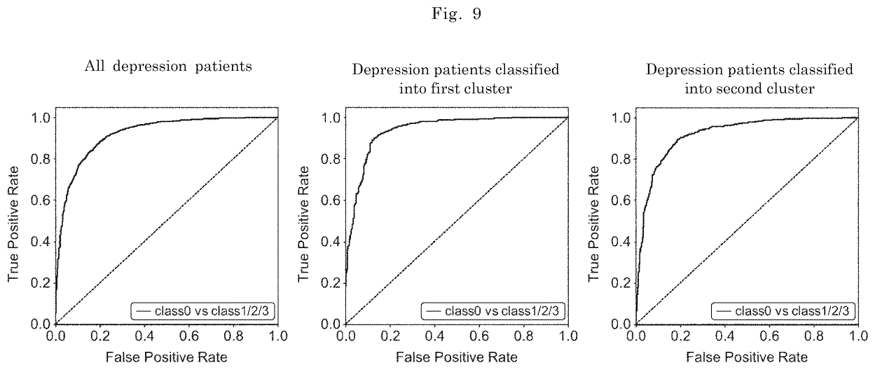

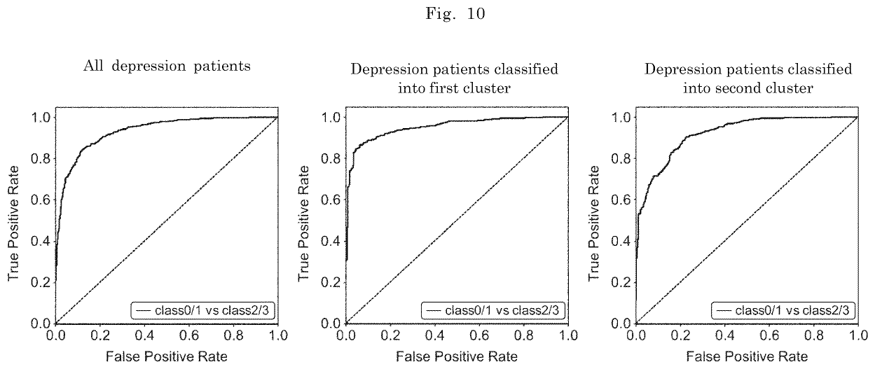

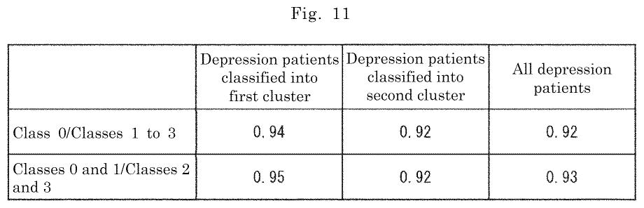

FIGS. 9 and 10 are diagrams illustrating a receiver operating characteristic (ROC) curve created based on the category predicted by the K6 score estimated using the created estimation model and the actual category classified based on the actual K6 score of the depression patient. Each ROC curve illustrated in FIG. 9 is a graph prepared by setting a ratio predicted as class 0 among depression patients who were actually class 0 as a true positive rate (TPR) and setting a ratio actually predicted as class 0 among depression patients of class 1, class 2 or class 3 as a false positive rate (FPR). Each ROC curve illustrated in FIG. 10 is a graph in which the proportion predicted as class 0 or class 1 among depression patients who were actually class 0 or class 1 is set as TPR, and the proportion predicted as class 0 or class 1 among depression patients who were actually class 2 or class 3 is set as FPR. Each of FIGS. 9 and 10 illustrates an ROC curve for all 89 depression patients, an ROC curve for depression patients classified into the first cluster, and an ROC curve for depression patients classified into the second cluster. FIG. 11 is a table showing values of an area under the ROC curve (AUC) calculated using each graph illustrated in FIGS. 9 and 10. As illustrated in FIG. 11, the AUC value was 0.92 or more in any graph, indicating that the accuracy of the prediction result was high.

### [Implementation Example by Software]

The function of the information processing apparatus 5 (hereinafter, referred to as a “apparatus”) is realized by a program for causing a computer to function as the apparatus, and a program for causing a computer to function as each control block of the apparatus (particularly, each unit included in the control unit 50).

In this case, the apparatus includes a computer including at least one control device (for example, a processor) and at least one storage device (for example, a memory) as hardware for executing the program. By executing the program by the control device and the storage device, the functions described in the above embodiments are realized.

The program may be recorded not temporarily but in one or a plurality of computer-readable recording media. The recording medium may or may not be included in the apparatus. In the latter case, the program may be supplied to the apparatus via any wired or wireless transmission medium.

Furthermore, some or all of the functions of the control blocks can be realized by a logic circuit. For example, an integrated circuit in which a logic circuit functioning as each control block is formed is also included in the scope of the present invention. In addition, for example, the functions of the control blocks can be realized by a quantum computer.

Furthermore, each processing described in each of the above embodiments may be executed by artificial intelligence (AI). In this case, the AI may operate in the control device, or may operate in another device (for example, an edge computer, a cloud server, or the like).

### SUMMARY

An information processing system according to a first aspect of the present invention comprises: a clustering unit that inputs, to a clustering model that classifies behavior patterns of a plurality of depression patients into a plurality of clusters, behavior record information in which time of behavior performed by an object person during a first period is recorded for each behavior type, and classifies behavior patterns of the object person into any of the plurality of clusters; a first feature amount generation unit that, based on measurement information including an activity amount and a sleep time of the object person measured during a second period, generates a first feature amount indicating an activity state of the object person for each partial period included in the second period; and an estimation unit that estimates, for each of the plurality of clusters, a magnitude of psychological stress of the object person after the second period based on object person information including attribute information of the object person, a cluster into which the object person is classified, and the first feature amount.

The information processing system according to a second aspect of the present invention, in the first aspect described above, may further comprise a second feature amount generation unit that generates a second feature amount indicating a behavior pattern of the object person for each partial period included in the second period from behavior record information in which time of behavior performed by the object person during the second period is recorded for each behavior type, in which the estimation unit may estimate, for each of the plurality of clusters, a magnitude of psychological stress of the object person after the second period based on the object person information, the cluster into which the object person is classified, the first feature amount, and the second feature amount.

In an information processing system according to a third aspect of the present invention, in the second aspect described above, the second feature amount may include a number of days during which a length of time of each behavior of the object person falls outside an upper limit or a lower limit of a predetermined confidence interval within the partial period.

In an information processing system according to a fourth aspect of the present invention, in the first aspect described above, the estimation unit may estimate the magnitude of the psychological stress by inputting the object person information and the first feature amount generated by the first feature amount generation unit to an estimation model prepared for a cluster into which a behavior pattern of the object person is classified by machine learning using teacher data in which the object person information and the first feature amount are used as explanatory variables and the magnitude of the psychological stress is used as an objective function.

In an information processing system according to a fifth aspect of the present invention, in the second or third aspect described above, the estimation unit may estimate the magnitude of the psychological stress by inputting the object person information, the first feature amount generated by the first feature amount generation unit, and the second feature amount generated by the second feature amount generation unit to an estimation model prepared for a cluster in which a behavior pattern of the object person is classified by machine learning using teacher data in which the object person information, the first feature amount, and the second feature amount are used as explanatory variables and the magnitude of the psychological stress is used as an objective function.

In an information processing system according to a sixth aspect of the present invention, in any one of the first to fifth aspects described above, the second period may be a plurality of weeks, and the partial period may be a plurality of days.

An information processing method according to a seventh aspect of the present invention is an information processing method by a computer, the information processing method comprising: a clustering step of inputting, to a clustering model that classifies behavior patterns of a plurality of depression patients into a plurality of clusters, behavior record information in which time of behavior performed by an object person during a first period is recorded for each behavior type, and classifies behavior patterns of the object person into any of the plurality of clusters; a first feature amount generation step of, based on measurement information including an activity amount and a sleep time of the object person measured during a second period, generating a first feature amount indicating an activity state of the object person for each partial period included in the second period; and an estimation step of estimating, for each of the plurality of clusters, a magnitude of psychological stress of the object person after the second period based on object person information including attribute information of the object person, a cluster into which the object person is classified, and the first feature amount.

The present invention is not limited to the above-described embodiments, and various modifications can be made within the scope indicated in the claims, and embodiments obtained by appropriately combining technical means disclosed in different embodiments are also included in the technical scope of the present invention.

### REFERENCE SIGNS LIST

5 Information processing apparatus
6, 8 Server
7, 7A, 10 Terminal device
20 Wearable terminal
51, 61, 71 Information acquisition unit
52, 62, 72 Clustering unit
53, 63, 73 First feature amount generation unit
54, 64, 74 Second feature amount generation unit
55, 65, 75 Estimation unit
100, 200, 300, 400 Information processing system

1. An information processing system comprising:
a clustering unit that inputs, to a clustering model that classifies behavior patterns of a plurality of depression patients into a plurality of clusters, behavior record information in which time of behavior performed by an object person during a first period is recorded for each behavior type, and classifies behavior patterns of the object person into any of the plurality of clusters;
a first feature amount generation unit that, based on measurement information including an activity amount and a sleep time of the object person measured during a second period, generates a first feature amount indicating an activity state of the object person for each partial period included in the second period; and
an estimation unit that estimates, for each of the plurality of clusters, a magnitude of psychological stress of the object person after the second period based on object person information including attribute information of the object person, a cluster into which the object person is classified, and the first feature amount.

2. The information processing system according to claim 1, further comprising a second feature amount generation unit that generates a second feature amount indicating a behavior pattern of the object person for each partial period included in the second period from behavior record information in which time of behavior performed by the object person during the second period is recorded for each behavior type,
wherein the estimation unit estimates, for each of the plurality of clusters, a magnitude of psychological stress of the object person after the second period based on the object person information, the cluster into which the object person is classified, the first feature amount, and the second feature amount.

3. The information processing system according to claim 2, wherein the second feature amount includes a number of days during which a length of time of each behavior of the object person falls outside an upper limit or a lower limit of a predetermined confidence interval within the partial period.

4. The information processing system according to claim 1, wherein the estimation unit estimates the magnitude of the psychological stress by inputting the object person information and the first feature amount generated by the first feature amount generation unit to an estimation model prepared for a cluster into which a behavior pattern of the object person is classified by machine learning using teacher data in which the object person information and the first feature amount are used as explanatory variables and the magnitude of the psychological stress is used as an objective function.

5. The information processing system according to claim 2, wherein the estimation unit estimates the magnitude of the psychological stress by inputting the object person information, the first feature amount generated by the first feature amount generation unit, and the second feature amount generated by the second feature amount generation unit to an estimation model prepared for a cluster in which a behavior pattern of the object person is classified by machine learning using teacher data in which the object person information, the first feature amount, and the second feature amount are used as explanatory variables and the magnitude of the psychological stress is used as an objective function.

6. The information processing system according to claim 1, wherein the second period is a plurality of weeks, and the partial period is a plurality of days.

7. An information processing method by a computer, the information processing method comprising:
a clustering step of inputting, to a clustering model that classifies behavior patterns of a plurality of depression patients into a plurality of clusters, behavior record information in which time of behavior performed by an object person during a first period is recorded for each behavior type, and classifies behavior patterns of the object person into any of the plurality of clusters;
a first feature amount generation step of, based on measurement information including an activity amount and a sleep time of the object person measured during a second period, generating a first feature amount indicating an activity state of the object person for each partial period included in the second period; and
an estimation step of estimating, for each of the plurality of clusters, a magnitude of psychological stress of the object person after the second period based on object person information including attribute information of the object person, a cluster into which the object person is classified, and the first feature amount.
